(19) United States
(12) Patent Application Publication
(10) Pub. No.: US 2020/0360027 A1
(43) Pub. Date: Nov. 19, 2020

(54) BLOOD OCCLUSION OR RESTRICTION CUFF

(71) Applicant: VALD PERFORMANCE PTY LTD, Newstead, Queensland (AU)

(72) Inventor: Joseph MARCUS, Mermaid Beach, New South Wales (AU)

(73) Assignee: VALD PERFORMANCE PTY LTD, Newstead, Queensland (AU)

(21) Appl. No.: 16/753,920

(22) PCT Filed: Oct. 5, 2018
(86) PCT No.: PCT/AU2018/051078

§ 371 Date: Apr. 6, 2020

(30) Foreign Application Priority Data
Oct. 6, 2017 (AU) 2017904033

(51) Int. Cl. A61B 17/135; A61B 8/06; A61B 8/04; A61B 8/08; A61B 5/1455; A63B 24/00

(52) U.S. Cl. CPC A61B 17/1355; A61B 8/06; A61B 8/04; A61B 2017/00132; A61B 5/14552; A63B 24/0075; A61B 8/488

(57) ABSTRACT

A remotely operated blood flow occlusion or restriction cuff for use as a muscle building device or a compact medical emergency reperfusion tourniquet. A module on the cuff housing an air pump, a controller operating the air pump and power supply. The inflation and deflation of the cuff controlled using a smart device in communication with the controller. In the preferred embodiment this is via a Smartphone app and Bluetooth protocol. The tourniquet operating in conjunction with a Doppler ultrasound and/or pulse oximeter to monitor arterial inflow.

### TECHNICAL FIELD

The present invention relates to the activity of body building, weight training as well as blood flow control in the medical field. In a preferred form, the invention comprises a smartphone application (app) operated fitness apparatus used to restrict venous blood flow to promote muscular strength and hypertrophy.

Furthermore, the invention can be used as a self-contained and wireless medical tourniquet to prevent critical blood loss and hypoxic tissue damage in emergency and traumatic life threatening situations.

The improvement of the invention is directed to removing the necessity of various inflation tubes and electrical cables associated with prior art cuffs and tourniquets, which not only restrict movement during training, but also require ancillary equipment such as external air pumps and monitoring devices required for their operation.

### BACKGROUND TO THE INVENTION

While the invention is directed to an improved self-contained cuff and tourniquet which dispenses with the requirement of lines, tubing or equipment currently associated with such devices, it is instructive to examine their present use and the state of the prior art.

### Blood Flow Restriction Training

According to conventional theory, to induce muscular hypertrophy, lifting weights with a minimum of 70% of one repetition maximum tests (1RM) is needed. It is accepted that intensities below this threshold lack the essential stimulus to produce muscular adaptations in the sarcoplasms and myofibrillars of muscle tissue (Peterson et al 2011). Recent studies, however, using relatively low loads of predicted 1RM as low as 20% combined with blood flow restriction (BFR) have been effective in eliciting muscular hypertrophy and strength similar to that achieved with higher loads (Abe et al 2005).

Prior art blood flow restriction training is a muscular hypertrophy and strength stimulus utilising the application of specially adapted pressure cuffs to the top of a limb e.g. an upper thigh and/or arm which are inflated to a predetermined pressure throughout the duration of the exercise (Abe et al 2005, Loenneke 2010). The pressure applied to the limb is strong enough to partially restrict the venous return of blood from peripheral muscle beds to the heart, but not strong enough to occlude arterial blood supply to the muscle region (Abe et al 2005, Loenneke 2010).

Blood flow restriction (BFR) training should not be confused with training under ischemic conditions. BFR does not induce ischemia within skeletal muscle, but rather promotes a state of blood pooling in the capillaries within the limb musculature as venous return is only slightly impaired and muscle bed perfusion under mean arterial pressure is maintained throughout the training load or exercise regimen (Abe et al 2005).

### Medical Tourniquets

The use of medical tourniquets is widely documented. In Outpatient clinics, where it is less critical, tourniquets usually comprise elastic or other manually operated bands. They do not allow for accurate pressure levels to be established and are used merely to occlude venous return or in the extreme, arterial blood supply to a limb to control blood loss. These simple occlusion cuffs, often connected to an analogue gauge, usually portable, comprise a hand operated pump which inflates an air cuff. While this is a basic, portable and low cost device, it cannot be used to accurately monitor the perfusion of a muscle bed. Simpler devices, such as blood flow restriction (BFR) bands, usually comprise elastic material to restrict blood flow. They are cheap and versatile but cannot gauge or retain pressure and invariably are not associated with monitoring systems.

In the intensive treatment of patients, where control of the circulatory system is vital, machine operated tourniquets connected to digital pressure monitors and pumps, allow for pressure to be accurately applied by controlled inflation of cuffs which impede blood flow. This is especially important where reperfusion of a damaged limb needs to occur periodically. Such tourniquets have to be programmed to deflate regularly to allow arterial inflow into the affected area to help mitigated the risk of tissue damage from hypoxia. Such prior art reperfusion tourniquets are generally associated with separate pump and control modules found more commonly in the accident and emergency (A & E) or intensive care units (ICU) of hospitals.

Relevant prior art medical tourniquets include the Kaatsu tourniquet, which is used widely in Japan, and comprises a machine with an electronic monitor system connected to air cuffs via plastic tubing. While it has the advantage of an electronic monitor which is relatively portable and accurate, it has external tubing, and therefore not ideal for dynamic exercise or mobile use. The Delphi Blood flow restriction cuff is a digital machine, connected to an intravenous (IV) pole with plastic tubing connected to an air cuff. It can monitor pressure applied to a limb accurately but is expensive, cumbersome, not portable, and needs to be connected to a mains power source. It also requires the use of external tubing which compromises the user's mobility and/or freedom of movement.

It is therefore an advantage to provide a versatile venous restriction or occlusion cuff without any attached tubing or cables for use in muscular strength and hypertrophy training, but which can also be used as an arterial reperfusion tourniquet to stop traumatic blood loss without tissue hypoxia at increased cuff pressures.

It is also an advantage that the invention is a standalone and relatively compact apparatus which allows for greater user mobility while training and which reduces the amount of space taken up in storage or transport.

Importantly, the invention seeks to provide the public with a commercial alternative and useful improvement over the prior art.

### SUMMARY OF THE INVENTION

In one aspect therefore, the invention resides in a remote controlled blood flow restriction cuff comprising:
an air bladder configured to be positioned around a limb;
a compact air pump located on the cuff to pressurise the air bladder;
a pressure sensor to sense bladder air pressure;
an electronic controller to control operation of the air pump; the controller adjustable for a predetermined pressure or pressure range, and receptive and responsive to signals from the pressure sensor;
a power supply located on the cuff to provide power to the air pump, controller and pressure sensor, and wherein inflation and deflation of the cuff can be controlled using a remote device in communication with the controller.

Preferably, the controller enables the cuff to be inflated to and retained at a pressure up to 350 mmHg. It will be obvious that an inflation pressure above that of a systolic pressure of a wearer will facilitate the cuff to be used as medical tourniquet to stop blood flow.

Preferably, the air pump is a battery operated air pump.

Preferably, the power supply is a rechargeable battery power supply.

Preferably, the air pump, power supply, pressure sensor, and controller are housed in a module proximal to the air bladder, which are all located on the cuff.

Preferably, the operation of the cuff is controlled remotely over a wireless protocol such as Bluetooth, associated with a Smart computer or phone application or other equivalent system.

Preferably, the pressure sensor detects pressure to the nearest 1 mmHg, wherein pressure in the cuff is maintained at the predetermined pressure level by the wireless controller operating the air pump responsive to signals from the pressure sensor.

Suitably, the controller accounts for variance in pressure due to limb movement for the duration required until the pressure is released from the cuff.

In another aspect, the invention resides in a method of using a remote controlled blood flow restriction cuff as herein described to build and/or strengthen muscle including the steps of:
a) applying the cuff to a limb;
b) initialising and connecting the cuff via a remote device or a Bluetooth protocol Smartphone application (app);
c) when connected, a user in response to a pre exercise questionnaire, inputs through the app, physiological data, age, height, medical history, gender, limb circumference size, body fat level, heart rate and blood pressure and any other requisite pre exercise information;
d) the user selects the most appropriate training program and range of cuff pressures expressed in mmHg;
e) the user sets a timer for the selected training program;
f) the user presses a start button of the remote device or the app;
g) the cuff inflates to a desired pressure;
h) the cuff through an electronic controller in response to a pressure sensor controlling an air pump maintains pressure at a specified range of cuff pressures, by inflating and deflating the cuff as necessary to account for limb movement;
i) once the timer has expired or the user stops the program, the cuff deflates, and
j) the Smartphone application records cuff pressures and duration in a user profile wherein data stored allows the user to generate training programs based upon the physiological data and stored cuff pressure data, as well as enabling the user to log sets and repetitions whereby the user can use the app for other wellness parameter tracking.

In another aspect, the invention resides in a method of using a remote controlled blood flow restriction cuff as herein described as a medical tourniquet including the steps of:
a) applying the cuff to an injured limb;
b) initialising the cuff operation via a remote device or a Bluetooth protocol Smartphone application (app);
c) monitoring arterial inflow via Doppler ultrasound;
d) increasing cuff pressure, via the device or app, to an arterial occluding pressure indicated by the Doppler signal;
e) automatically maintaining arterial occluding cuff pressure measured by the pressure sensor and also inflating or deflating the cuff in response to the Doppler signal;
f) rating the level of traumatic injury to a limb via the remote device or app, wherein a reperfusion program is selected to automatically allow brief periods of cuff deflation and re-inflation for reperfusion of the limb thereby mitigating risk of unnecessary hypoxic tissue damage;
g) monitoring arterial inflow via the Doppler ultrasound wherein the cuff is only deflated and re-inflated in response to the Doppler signal to minimise blood loss in accordance with the reperfusion program, wherein
h) once full medical or hospital intervention can be provided, the cuff can then be deflated and removed under medical supervision.

Suitably, where available or desired, a pulse oximeter can be used in the alternative or in conjunction with a Doppler ultrasound to monitor arterial inflow.

It will be obvious, the advantage of using oximetry would be a readily obtainable pulse rate and a blood oxygen saturation level.

The present invention is a digital tourniquet device with no external wires or tubing, and is a fully self-enclosed pneumatic air cuff. They are designed to be used as medical tourniquets during trauma events to stop blood flow and to be used in fitness during blood flow restriction training.

They are blood flow restriction devices which are digital, wireless, and pneumatic cuffs, to be worn on the upper limbs and lower limbs. The blood flow restriction or occlusion device of the present invention is electronic, completely wireless and tubeless design that includes an integrated power supply. The device is self-contained, but interacts with an untethered controller that is adapted to be worn on the limbs of a user.

The device measures individualised pressure zones of a user, which are based on personalised physiological data, and maintains a restriction pressure that is exerted upon the limb, which is applied consistently in real time, and accounts for movement of limb. This means that the device makes adjustments to increase/decrease pressure in order to maintain the preselected pressure in accordance with the individualised pressure zone of the user.

The physiological data can include blood flow, pulse rate, blood pressure, limb occlusion pressure (LOP) and arterial occlusion pressure (AOP). The physiological data for blood flow and pulse rate are collected via the following methods, which can be determined by integrated and off the shelf devices coupled with smartphone apps;
Photo plethysmography
Bio impedance
Near Infrared Spectroscopy (NIRS)
Doppler Ultrasound

### EXAMPLES

### Arterial Occlusion Pressure

Arterial occlusion pressure (AOP) is a measure of the cuff pressure required to maintain a bloodless surgical field.

### The Device of the Present Invention Performs the Following Calculations:—

1. Measures Systolic Blood Pressure (SBP)
2. The user then inputs their limb circumference data into the controller
3. The circumference data of the limb is used to calculate the corresponding estimated KTP coefficient as based on the following table:—

TABLE 2
Tissue padding coefficients based on limb circumferences.^{[25]}

| Extremity | Estimated |
|---|---|
| circumferences (cm) | K_{TP} |
| 20 | 0.91 |
| 21 | 0.90 |
| 22 | 0.89 |
| 23 | 0.88 |
| 24 | 0.87 |
| 25 | 0.86 |
| 26 to 27 | 0.85 |
| 28 | 0.84 |
| 29 | 0.83 |
| 30 to 31 | 0.82 |
| 32 to 33 | 0.81 |
| 34 | 0.80 |
| 35 to 36 | 0.79 |
| 37 to 38 | 0.78 |
| 39 to 40 | 0.77 |
| 41 to 43 | 0.76 |
| 44 to 45 | 0.75 |
| 46 to 48 | 0.74 |
| 49 to 51 | 0.73 |
| 52 to 54 | 0.72 |
| 55 to 57 | 0.71 |
| 58 to 60 | 0.70 |
| 61 to 64 | 0.69 |
| 65 to 68 | 0.68 |
| 69 to 73 | 0.67 |
| 74 to 75 | 0.66 |

K_{TP} = Tissue padding coefficient.

4. The arterial occlusion pressure is calculated according to the following:—

$$\frac{Systolic Blood Pressure + 10}{KTP coefficient}$$

5. Then the user is then provided four options to select;
   50% of total pressure
   60% of total pressure
   70% of total pressure
   80% of total pressure
6. The cuff is then inflated and maintained at the selected % of AOP, and the device keeps this pressure constant, allowing for adjustments with limb/body movement.

### Limb Occlusion Pressure

Limb occlusion pressure or LOP can be defined as the minimum pressure required to stop the flow of arterial blood into the limb distal to the cuff. LOP is determined by gradually increasing tourniquet pressure until distal blood flow is interrupted.

The risk of tourniquet related complications can be significantly reduced by measuring the LOP and selecting cuff inflation pressures accordingly. Best practice recommends that optimal cuff pressure should be based on the patient's LOP. The tourniquet pressure should be minimized, lower pressures are thought to prevent injury of normal tissue.

The device of the present invention calculates the limb occlusion pressure (LOP) in accordance with the following steps:—
1. The user opens the controller
2. The user places the device on a limb
3. The device uses the following methods to monitor blood flow in the user's extremity;
   Photoplethysmography
   Bioimpedance
   Near Infrared Spectroscopy (NIRS)
   Doppler ultrasound
4. The device inflates in increments of 5-10 mmhg until pulse stops
5. The device senses at what pressure level pulse rate is no longer detected using aforementioned methods of measuring blood flow and pulse
6. The point at which minimal pressure required to stop blood flow to extremity to limb is called Limb Occlusion Pressure (LOP)
7. The blood flow restriction parameters are based upon LOP
8. The cuff will inflate to desired percentage of LOP 50-80% based on user selection
9. The user begins use of device with selected pressure of LOP
10. The cuff is then inflated and is maintain at selected % of LOP, device keeps pressure constant in this zone, allowing for adjustments with limb/body movement.

### LOP Measurement

Individual limb occlusion pressure will be measured manually by (a) palpation, and (b) Doppler ultrasound (c) using a distal photo plethysmography sensor (d) NIRS (e) bio impedance.

Tourniquet cuff is automatically inflated and utilizes aforementioned probes to detect arterial pulsations in limb at level of cuff and distal to the tourniquet cuff.

### Benefits of Calculating LOP

When measured correctly in applying LOP in BFR using surgical grade automated devices, LOP helps in delivering optimum results in rehabilitation by personalizing blood flow restriction training for rehabilitation of elderly, improving performance of injured athletes and in patients recovering from major surgical procedures such as knee arthroscopy.

The devices of the present invention are inflated to a pressure of between 0-350 mmhg. There is an on-board module housing the battery operated air pump. The on-board pressure sensor or monitor detects the desired pressure to the nearest 1 mm Hg. Pressure is maintained at a desired pressure level for the duration needed and is corrected for variance in pressure with limb movement. Pressure is then released by the user. The device is preferably controlled via Bluetooth or wireless controller with accompanying Smartphone application. The devices are to be used in conjunction with and without low load resistance training to increase muscular size and strength comparatively to exercising with higher loads.

Exercising at necessary intensity can cause injury, and people who would benefit most from exercise often cannot participate e.g. injured or sick patients. Typically, to achieve positive muscular adaptations, individuals have to exercise at above 80% of their maximum level. According to literature, however, when low load exercise of 10-30% of maximum is combined with blood flow restriction, the same results to non-blood restriction training can be achieved in comparative time frames.

Currently on the market, there exists two extreme versions of the prior art. These include cumbersome, wired, heavy, medical devices, which are not portable and easily used, or expensive elastic tourniquets, which are not accurate and offer no physiological data, and which are prone to abuse by over tightening.

### BRIEF DESCRIPTION OF THE DRAWINGS

Preferred embodiments of the invention will now be described, by way of example only, with reference to the accompanying drawings in which:

FIGS. 1 to 4 show various views of a preferred blood flow restriction cuff in accordance with the invention.

FIG. 5 shows the embodiment of FIGS. 1 to 4 in use as an exercise cuff.

FIG. 6 shows the embodiment of FIGS. 1 to 4 in use as a medical tourniquet.

### DETAILED DESCRIPTION OF PREFERRED EMBODIMENTS

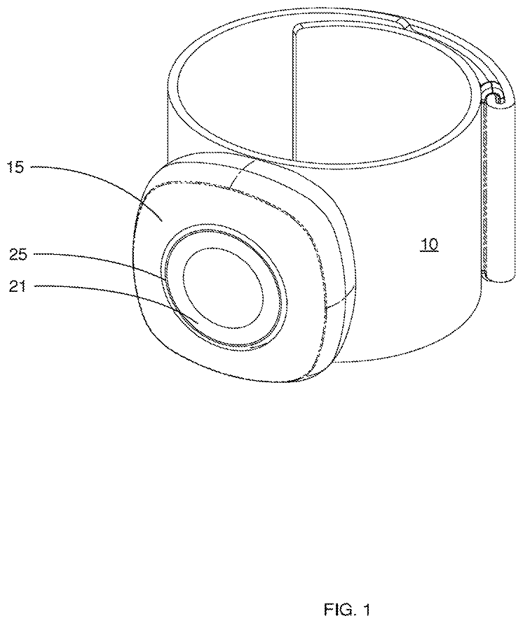

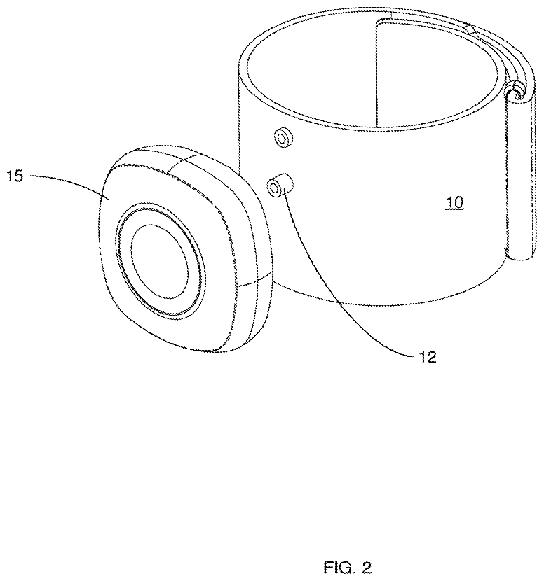

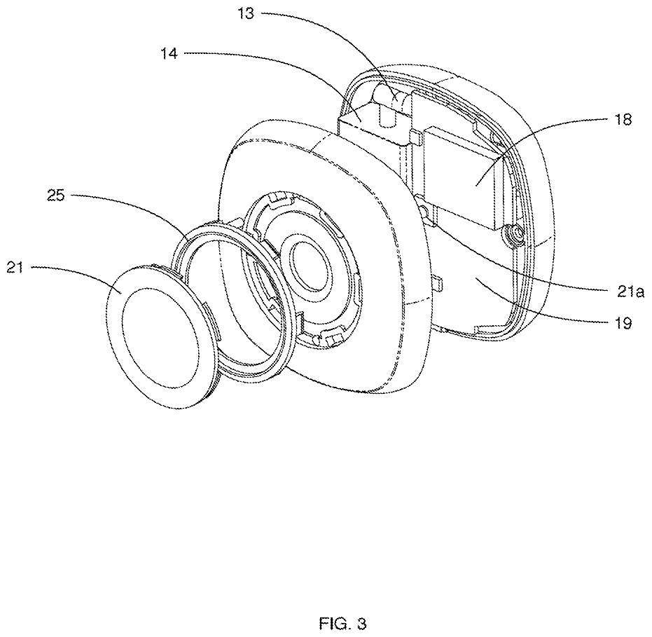

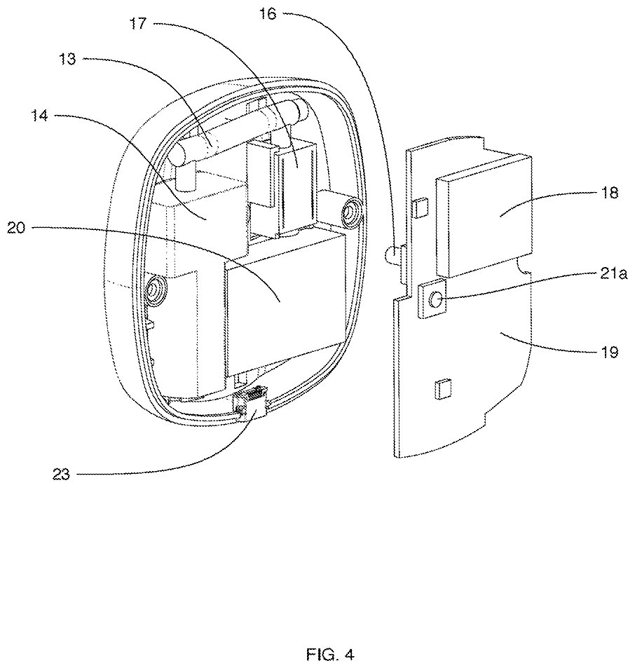

FIGS. 1 to 4 show views of a preferred remote controlled blood flow restriction cuff 10 according to the invention. Cuff 10 is configured to be positioned around a limb (not shown). Compact air pump 14 in module 15 pressurises the air bladder (not visible as sewn inside the cuff) via tube 13 connected to valve 17 which supplies air to the bladder through air inlet 12. Pressure sensor 16 on PCB board 19 senses the air pressure in the air bladder (not visible as sewn inside the cuff). Microprocessor based electronic controller 18 on PCB board 19 with reset button 21 and reset switch 21a controls operation of the air pump 14 and is adjustable for a predetermined pressure or pressure range. Microprocessor controller 18 is receptive and responsive to signals received from pressure sensor 16.

Power supply 20 is located on the cuff to power the air pump 14 and the controller 18, wherein inflation and deflation of the air bladder is controlled using a remote device such as a Smartphone (not shown) in communication with the controller 18.

As previously mentioned preferably, controller 18 enables the cuff to be inflated to and retained at a pressure up to 350 mmHg. Which will be an inflation pressure above that of systolic pressure of most users which will also facilitate the cuff to be used as medical tourniquet to stop arterial blood loss. Air pump 14 is battery operated and power supply 20 is a rechargeable battery power supply.

As is shown, air pump 14, power supply 20, pressure sensor 16, and controller 18 are housed in a module 22 on cuff 10.

In the preferred embodiment, operation of the cuff 10 is controlled remotely over a wireless protocol such as Bluetooth, associated with a Smart computer or phone application or other equivalent wireless system for example a USB programmed memory stick (not shown) inserted in USB port 23.

Preferably, pressure sensor 16 detects pressure to the nearest 1 mmHg, wherein pressure in the air bladder is maintained at the predetermined pressure level by controller 18 operating the air pump 14 responsive to signals from pressure sensor 16.

Suitably, controller 18 accounts for variance in pressure due to limb movement for the duration required until the pressure is released from the air bladder. Circular LED light 25 shows that the power supply 20 has been turned on.

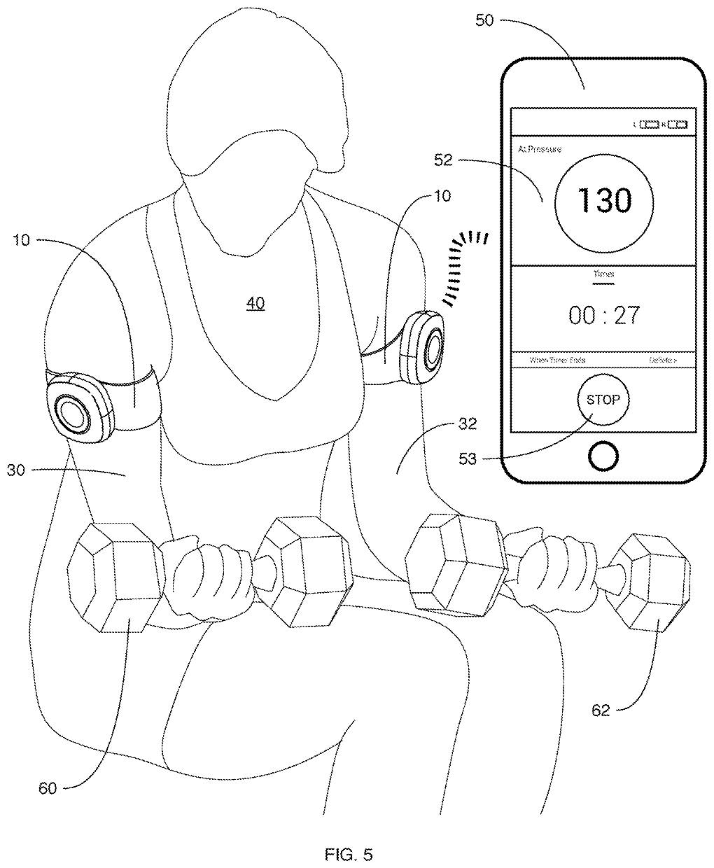

FIG. 5 shows the embodiment of FIGS. 1 to 4 in use as training cuffs 10 to build and strengthen muscle, in this case, the upper arms 30, 32 by lifting dumbbells 60, 62.

The cuffs 10 are applied to the upper arms 30, 32. User 40 initialises and connects with the controller of the cuff via a smartphone 50 with an application (app) 52 running on a Bluetooth protocol.

When connected, the user 40 in response to a pre exercise questionnaire, inputs through the app 52, physiological data, age, height, medical history, gender, limb circumference size, body fat level, heart rate and blood pressure and any other requisite pre exercise information required.

The user 40 then selects the most appropriate training program and a range of cuff pressures and sets a timer for the selected training program. The user presses a start button 53 displayed by the app.

The cuffs 10 inflate to the desired pressure and through the electronic controller in response to a pressure sensor controls the air pump which maintains pressure by inflating and deflating the cuff as necessary to account for any limb movement.

Once the timer has expired or the user stops the program, the cuff deflates, and Smartphone app 52 records cuff pressures and duration in a user profile.

As the smart phone application monitors physiological data continuously and wirelessly via Bluetooth (arterial inflow, pulse rate and oxygen saturation) this physiological data is put through an algorithm in conjunction with age, weight, limb circumference, and lean fat-free mass. This information is used to create a personalised user profile to establish the safest pressure parameters as a percentage of ‘Limb Occlusion Pressure’ and is customised to each user's needs as a hypertrophy/strength inducing blood flow restriction training device or as a life preserving medical tourniquet device (see below). In the training environment, the smart phone application will store data per session, pressure used, duration, and physiological data which can be used to create a customised user blood flow restriction exercise program.

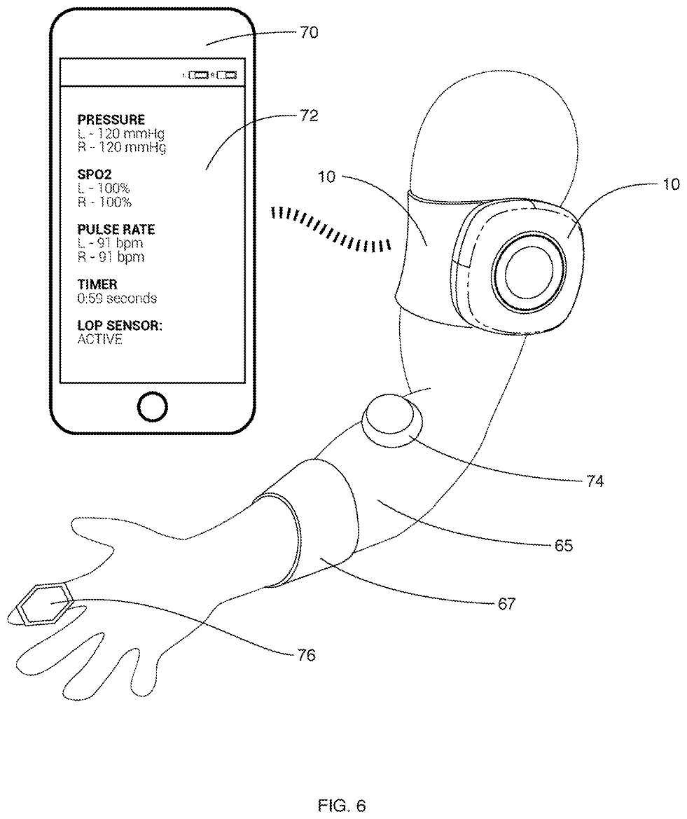

FIG. 6 shows the embodiment of FIGS. 1 to 4 in use as a medical tourniquet.

Cuff 10 is applied to an injured limb 65 with a wound dressed by bandage 67. A user initialises and connects with the controller of the cuff via a smartphone 70 with an application (app) 72 running on a Bluetooth protocol. Arterial inflow to the limb 65 is monitored via a Doppler ultrasound pickup 74. The user then increases cuff pressure, via the app, to an arterial occluding pressure indicated by the attenuation of the Doppler signal. The controller automatically maintains the arterial occluding cuff pressure measured by the pressure sensor by inflating or deflating the cuff in response to the Doppler signal.

The user rates the level of traumatic injury to a limb via the app, wherein a reperfusion program is selected to instruct the controller to enable brief periods of cuff deflation and re-inflation for reperfusion of the limb thereby mitigating the risk of unnecessary hypoxic tissue damage.

The controller monitors arterial inflow wherein the cuff is only deflated and re-inflated in response to the Doppler signal to minimise blood loss in accordance with the reperfusion program. A digital pulse oximeter 76 can also be used to monitor blood oxygen saturation and heartrate.

Once full medical or hospital intervention can be provided, the cuff can then be deflated and removed under medical supervision.

The medical tourniquet cuff and application utilises a smart phone or wireless interface display unit driven system of blood flow restriction achieved by using completely self-contained digital air pneumatic cuffs to impede venous arterial return through a self-regulating pressure sensor including blood flow restriction or occlusion monitoring via continuous wave Doppler ultrasound and/or pulse oximetry.

Continuous wave Doppler ultrasound has the following main function:

By sending and receiving ultrasound waves through the skin located over an artery or heart, when the waves are reflected from a moving object, such as blood passing through an artery, the reflected frequency changes slightly. This change is then analysed by the electronics of the Doppler ultrasound unit and converted into a digital display of the cardiac output and heart rate.

A pulse oximeter has two main functions, which are as follows:

To provide an audible signal of pulsed blood flow, similar to that of a Doppler ultrasound. When an artery is occluded by a pressure cuff, there is a loss of signal from the oximeter; and to measure the oxygen saturation of haemoglobin in blood or tissue.

Continuous wave Doppler ultrasound and the pulse oximetry are used in the measurement of pulse wave transit time distal to position of the cuff on the limb. Pulse wave transit time is defined as the time required for an arterial pulse wave to propagate along a fixed path, which is used to determine ‘Limb Occlusion Pressure’. This measurement can be made by monitoring the pulse at a point distal to the application point of the tourniquet. Once the distal pulse rate is absent at the most minimal arterial pressure, this is determined to be the ‘Limb Occlusion Pressure’.

The following include examples of procedures which can be adopted in the use of the invention.

First time use and creation of personalised Smartphone Application User Profile. Explain the procedure to the patient; Ensure he/she is lying comfortably in a semi-recumbent position.

### Stage 1—Measuring Upper Limb Pressure Using the Pulse Oximeter.

Place an appropriate size blood-pressure cuff around the patient's upper arm;

Place the pulse oximeter sensor on any finger (FIG. 1a). When the sensor is placed on the digit, the oximeter will display two numbers: the first represents the patient's heart rate, the second the percentage of circulating oxygenated haemoglobin. Pulse blood flow is also displayed on the oximeter, either by a waveform or by a column of lights;

Record a baseline reading. Inflate the cuff to 60 mmHg, then inflate it in 10 mmHg increments, allowing approximately 10 seconds between these increments. Once the pressure reaches 100 mmHg, the incremental changes can be increased to 20 mmHg;

Record the pressure reading that is one below the point where the audible or pulse signal is lost on the pulse oximeter; for example, if the signal is lost at 180 mmHg, record a pressure of 160 mm Hg;

Repeat the measurement on the other arm, then calculate the ‘Limb Occlusion Pressure’ by using the higher of the two readings

### Stage 2—Measuring Lower Limb Pressure Using the Pulse Oximeter.

Place an appropriate size cuff around the largest most proximal thigh region.

Place the oximeter sensor on one of the first three toes (FIG. 1b).

Inflate the cuff as outlined in Stage 1, and record the pressure at which the signal is lost. Repeat the measurement on the other leg, then calculate the ‘Limb Occlusion Pressure’ index by using the higher of the two readings.

The pressure at which the arterial pulse is stopped corresponds to the minimum tourniquet cuff pressure to occlude the underlying arteries or ‘Limb Occlusion Pressure’ at that time.

After the ‘Limb Occlusion Pressure’ is identified, this data is fed through the accompanying Smartphone application. Working pressures for blood flow restriction are personalised using the ‘Limb Occlusion Pressure’ data gathered. Each user's physiological details are stored in the Smartphone application. The micro processer (or controller) and the pressure sensor/pump implements the calculated pressure target as a percentage of the ‘Limb Occlusion Pressure’. This personalised approach ensures only the most minimal blood impedance pressures are applied, helping to mitigate the risks associated with tourniquet application. This ensures arterial inflow into a limb but restricts venous return, which leads to a cascade of physiological benefits as earlier mentioned.

The present invention allows individuals to engage in blood flow restriction training more safely and accurately. It has all the safety and physiological data of expensive wired medical devices but with the added freedom of having all electronics housed in an on-board module with no external tubing or wiring.

It is worn on the upper and lower limbs. Preferably, the cuffs are 5 cm or 10 cm wide pneumatic cuffs comprising of an outer cuff material of leather or silicone with airbag contained between the material layers. The cuffs are preferably applied using a ratchet or buckle or Velcro loop fastening system. The pneumatic cuff for an upper limb is 25 cm to 50 cm length and for a lower limb is 50 to 75 cm length.

The airbag is connected to the module unit with a two way valve which can be closed to allow pressure to be held and maintained at a desired pressure level with the pressure sensor and controller maintaining this pressure irrespective of limb movement by making small adjustments in air pressure to allow maintenance of the desired pressure. The cuff module contains an on-board battery, pressure sensor and air pump all housed in a plastic casing which allows the airbag to be inflated and to increase pressure (mmHg) in the airbag. This increase in pressure in mediated by the on-board sensor which increases pressure to a predetermined level between 0-350 mmHg and maintains the set pressure for the duration of use.

The system then allows pressure to be released when desired by the user. Communication with the device is via Bluetooth or an equivalent wireless protocol. The device will have accompanying smart phone application or a wireless remote control unit. There is no external tubing or wiring. The device is completely free and is only attached to a limb by the releasable fastening system.

In summary, the invention can be described as a wireless operated tubeless air pneumatic cuff system with a dual purpose role in blood flow restriction training and as a portable tourniquet for medical emergencies. As a wireless operated tourniquet, it enables pressure exerted on limb to be maintained at a desired level through an inbuilt pressure sensor and controller. The main advantage of the device is that it is completely wireless, portable and self-contained without requiring extraneous tubing connected to ancillary equipment. All electronics and the air bladder are encased within the cuff itself. The device has application in the medical field by being used as a digital medical tourniquet to stop traumatic blood loss at higher cuff pressures as well as in the fitness industry in conjunction with low load resistance training to increase muscle strength and hypertrophy.

It is conveniently operated via a Smartphone application which reacts to physiological data including arterial pulse rate and blood oxygen saturation levels to create a user profile which can then be programmed to automatically control occlusion pressure levels according to customised training protocols.

In this specification, unless the context clearly indicates otherwise, the term “comprising” has the non-exclusive meaning of the word, in the sense of “including at least” rather than the exclusive meaning in the sense of “consisting only of”. The same applies with corresponding grammatical changes to other forms of the word such as “comprise”, “comprises” and so on.

It will be apparent that obvious variations or modifications may be made which are in accordance with the spirit of the invention and which are intended to be part of the invention, and any such obvious variations or modifications are therefore within the scope of the invention.

### REFERENCES

Abe, T., Yasuda, T., Midorikawa, T., Sato, Y., Kearns, C. F., Inoue, K., & Ishii, N. (2005a). Skeletal muscle size and circulating IGF-1 are increased after two weeks of twice daily “KAATSU” resistance training. International Journal of KAATSU Training Research, 1(1), 6-12.
Loenneke, J. P., Wilson, G. J., & Wilson, J. M. (2010). A mechanistic approach to blood flow occlusion. Int J Sports Med, 31(1), 1-4.
Peterson, M. D., Pistilli, E., Haff, G. G., Hoffman, E. P., & Gordon, P. M. (2011). Progression of volume load and muscular adaptation during resistance exercise. European journal of applied physiology, 111(6), 1063-1071.
Estimation of limb occlusion pressure for surgical tourniquets based on the measurement of Arterial Pulse Wave Transit Time. Marko, Alexei John. 1994. Vancouver: University of British Columbia Library.

1. A remote controlled blood flow restriction cuff comprising:
an air bladder configured to be positioned around a limb;
a compact air pump located on the cuff to pressurise the air bladder;
a pressure sensor to sense bladder air pressure;
an electronic controller to control operation of the air pump; the controller adjustable for a predetermined pressure or pressure range, and receptive and responsive to signals from the pressure sensor;
a power supply located on the cuff to provide power to the air pump, controller and pressure sensor, and wherein inflation and deflation of the cuff can be controlled using a remote device in communication with the controller.

2. The restriction cuff according to claim 1 wherein the controller enables the cuff to be inflated to and retained at a pressure up to 350 mmHg.

3. The restriction cuff according to claim 1 wherein the air pump is a battery operated air pump.

4. The restriction cuff according to claim 1 wherein the power supply is a rechargeable battery power supply.

5. The restriction cuff according to claim 1 wherein the air pump, power supply, pressure sensor, and controller are housed in a module proximal to the air bladder, which are all located on the cuff.

6. The restriction cuff according to claim 1 wherein, the operation of the cuff is controlled remotely over a wireless protocol such as Bluetooth, associated with a Smart computer or phone application (app).

7. The restriction cuff according to claim 1 wherein the pressure sensor detects pressure in the cuff which is maintained at the predetermined pressure level by the controller operating the air pump responsive to signals from the pressure sensor.

8. The restriction cuff according to claim 1 wherein the controller receptive to signals from the pressure sensor accounts for variance in pressure due to limb movement.

9. The restriction cuff according to claim 1 wherein the cuff is positioned around a limb by a releasable fastening means.

10. The restriction cuff according to claim 1 wherein the wherein, the operation of the cuff is controlled with a smart phone application (app) and wherein the app includes customised programs for muscle building and strength, a timer, pressure controls, emergency stop, and tracks relevant physiological data with ability to store exercise variable data such as repetitions, sets and pressure history.

11. The restriction cuff according to claim 1 used as a medical tourniquet which allows pressure to be electronically increased and decreased via a smart phone application, wherein pressure is set at a desired level, and the pressure sensor and air pump make micro adjustments to keep cuff pressure constant at the desired level.

12. The restriction cuff according to claim 1 worn on a proximal upper limb (e.g. upper arm) and a proximal lower limb (e.g. upper thigh).

13. The restriction cuff according to claim 1 wherein the cuff is a leather or nylon or silicone 5 or 10 cm cuff with a ratchet or buckle or loop Velcro fastening system.

14. A method of using a remote controlled blood flow restriction cuff to build and/or strengthen muscle including the steps of
a) applying the cuff to a limb;
b) initialising and connecting the cuff via a remote device or a Bluetooth protocol Smartphone application (app);
c) when connected, a user in response to a pre exercise questionnaire, inputs through the app, physiological data, age, height, medical history, gender, limb circumference size, body fat level, heart rate and blood pressure and any other requisite pre exercise information;
d) the user selects the most appropriate training program and range of cuff pressures expressed in mmHg;
e) the user sets a timer for the selected training program;
f) the user presses a start button of the remote device or the app;
g) the cuff inflates to a desired pressure;
h) the cuff through an electronic controller in response to a pressure sensor controlling an air pump maintains pressure at a specified range of cuff pressures, by inflating and deflating the cuff as necessary to account for limb movement;
i) once the timer has expired or the user stops the program, the cuff deflates, and
j) the Smartphone application records cuff pressures and duration in a user profile wherein data stored allows the user to generate training programs based upon the physiological data and stored cuff pressure data, as well as enabling the user to log sets and repetitions whereby the user can use the app for other wellness parameter tracking.

15. A method of using a remote controlled blood flow restriction cuff as a medical tourniquet including the steps of:
a) applying the cuff to an injured limb;
b) initialising the cuff operation via a remote device or a Bluetooth protocol Smartphone application (app);
c) monitoring arterial inflow via Doppler ultrasound;
d) increasing cuff pressure, via the device or app, to an arterial occluding pressure indicated by the Doppler signal;
e) automatically maintaining arterial occluding cuff pressure measured by the pressure sensor and also inflating or deflating the cuff in response to the Doppler signal;
f) rating the level of traumatic injury to a limb via the remote device or app, wherein a reperfusion program is selected to automatically allow brief periods of cuff deflation and re-inflation for reperfusion of the limb thereby mitigating risk of unnecessary hypoxic tissue damage;
g) monitoring arterial inflow via the Doppler ultrasound wherein the cuff is only deflated and re-inflated in response to the Doppler signal to minimise blood loss in accordance with the reperfusion program, wherein
h) once full medical or hospital intervention can be provided, the cuff can then be deflated and removed under medical supervision.

16. The method of claim 15 including the step of using a pulse oximeter in the alternative, or in conjunction with, the Doppler ultrasound to monitor the arterial inflow.
